(19) United States
(12) Patent Application Publication
(10) Pub. No.: US 2026/0137435 A1
(43) Pub. Date: May 21, 2026

(54) FREQUENCY CONTROL OF IMPACT MECHANISM

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Kevin W. Belew, Hernando, MS (US); Abel C. Kim, Cordova, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/119,498

(22) PCT Filed: Nov. 9, 2023
(86) PCT No.: PCT/US2023/037096

§ 371 Date: Apr. 9, 2025

Related U.S. Application Data
(60) Provisional application No. 63/425,404, filed on Nov. 15, 2022.

(51) Int. Cl. A61B 17/92

(52) U.S. Cl. CPC A61B 17/92

(57) ABSTRACT

An orthopedic surgical instrument or impactor system and associated methods thereof. In some examples, the impactor system includes an impact mechanism coupled to rotational-to-linear conversion mechanism, which is coupled to a motor assembly using an electromagnetic clutch. The electromagnetic clutch transmits rotational motion generated by the motor assembly to the rotational-to-linear conversion mechanism for conversion of the rotational motion to translational motion to cause one or more cyclic translations of the impact mechanism during a predetermined period of time or cycle. The cyclic translations of the impact mechanism include oscillatory forward and reverse movements. The electromagnetic clutch is configured to temporarily engage and disengage the motor assembly and the rotational-to-linear conversion mechanism.

### CROSS-REFERENCE TO RELATED APPLICATIONS

This is a non-provisional of, and claims the benefit of the filing date of, U.S. provisional patent application number 63/425,404, filed Nov. 15, 2022, entitled “Frequency Control of Impact Mechanism”, the entirety of which application is incorporated by reference herein.

### TECHNICAL FIELD

The present disclosure is generally directed to an orthopedic surgical instrument, and, more specifically, to a powered, light-weight orthopedic surgical instrument or impactor capable of implementing a frequency control mechanism to impacts.

### BACKGROUND

Orthopedic surgical procedures such as, for example, hip procedures, knee procedures, shoulder procedures, etc., have become common place in today's society. For example, total hip arthroplasty or hip replacement is a well-known procedure for repairing damaged bone (e.g., a damaged hip). During a total hip arthroplasty, an acetabular system may be implanted into a patient's acetabulum. In addition, and/or alternatively, a femoral implant may be implanted into a patient's femur. During the surgical procedure, the patient's bone typically needs to be prepared to receive the orthopedic implant. For example, a surgical tool such as, for example, an orthopedic broach, rasp, cutting tool, etc. (terms used interchangeably herein without the intent to limit or distinguish) may be used to prepare an inner surface of a patient's intramedullary canal to receive an orthopedic implant such as, for example, a femoral hip prosthesis, an intramedullary nail, etc. The preparation of the intramedullary canal by the surgeon is designed to insure a proper fit between the patient's femur and the implant. In addition, the orthopedic implant such as, for example, the acetabular cup, may need to be impacted into proper position. Moreover, during removal of the broach from the patient's intramedullary canal, the broach may become struck within the patient's intramedullary canal.

Various surgical tools have been developed to assist surgeons during orthopedic procedures to place and/or remove various objects. For example, mallets are frequently used to apply an impacting force on the orthopedic tool (e.g., broach) to remove bones or other implanted objects. In addition, the mallets may be used to assist in removing the orthopedic tool (e.g., broach) if it becomes struck during the surgical procedure. Moreover, mallets may also be used to assist the surgeon with inserting the implant (e.g., mallets have been used to insert femoral nails, intramedullary nails, acetabular cups, etc.).

Currently, various steps during an orthopedic surgery involve a manual mallet impaction, such manual impactions typically cause fatigue and repeated-use injuries to the surgeon, as well as cause undesired variability during surgery. One existing solution to this problem includes a handheld powered impactor device also referred to herein as orthopedic impactors, impactors, or slap hammers (terms used interchangeably herein without the intent to limit or distinguish), however, many such impactor devices are either too large and heavy or do not apply enough impact force, thereby resulting in missteps during surgery, damage to bones, or other failures.

Orthopedic impactors have been developed to assist with driving a surgical tool or implant into the patient's bone, and to remove a struck or lodged surgical tool or implant from the patient's bone. However, these impactors also suffer from various issues. Some existing impactors have a large overall weight and fail to apply reliable impact force, in particular, application of impact force by such impactors is typically dependent on frequency of impact and vice versa. These issues typically cause substantial wear and tear on the components of the impactor as well as provide inconsistent results during medical procedures.

Thus, it would be beneficial to provide an impactor system, where force of impacts may be controlled independent of the frequency of impacts. It is with respect to these and other considerations that the present disclosure may be useful.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In one example, the current subject matter relates to an orthopedic impactor system. The impactor system may include an impact mechanism configured to be coupled to rotational-to-linear conversion mechanism, which may be coupled to a motor assembly using an electromagnetic clutch. The electromagnetic clutch may be configured to transmit rotational motion generated by the motor assembly to the rotational-to-linear conversion mechanism for conversion of the rotational motion to translational motion to cause one or more cyclic translations of the impact mechanism during a predetermined period of time or cycle. The cyclic translations of the impact mechanism may include oscillatory forward and reverse movements. The electromagnetic clutch may be configured to temporarily engage and disengage the motor assembly and the rotational-to-linear conversion mechanism.

In any preceding or subsequent examples, the system may also include a flywheel coupled to the motor assembly and configured to transmit rotational motion to the rotational-to-linear conversion mechanism upon engagement of the electromagnetic clutch.

In any preceding or subsequent examples, the impact mechanism may include one or more sensors configured to detect a position of the impact mechanism. Based on the position of the impact mechanism, the system may be configured to engage and/or disengage the electromagnetic clutch.

In any preceding or subsequent examples, each engagement and/or disengagement of the electromagnetic clutch may be determined based on at least one of the following: a length of a cycle of operation of the impact mechanism, a frequency of operation of the impact mechanism, a number of engagement and/or disengagement states during the cycle of operation of the impact mechanism, and any combination thereof.

In any preceding or subsequent examples, the impact mechanism may be configured to operate in a forward translational motion and a reverse translational motion.

In the forward translational motion, the electromagnetic clutch may be configured to provide an engagement of the motor assembly during a forward translational motion of the impact mechanism from a forward initial position to a forward impact position and configured to disengage the motor assembly upon the impact mechanism returning to the forward initial position.

In the reverse translational motion, the electromagnetic clutch may be configured to provide an engagement of the motor assembly during a reverse translational motion of the impact mechanism from a reverse initial position to a reverse impact position and configured to disengage the motor assembly upon the impact mechanism returning to the reverse initial position.

Examples of the present disclosure provide numerous advantages. For example, the orthopedic impactor enables a medical professional (e.g., an orthopedic surgeon, a doctor, etc.) to accurately and safely apply force to an orthopedic implant or a surgical tool. In particular, the current subject matter may be configured to allow such medical professional to control a frequency of impact (e.g., during forward and/or reverse movements of the impactor) so that the impacts occur at uniform intervals, where the impact force, as delivered by the impactor, is determined by the rotational speed of the motor of the impactor. This alleviates substantial wear-and-tear on the operational components of the impactor, while allowing consistent impact application.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings,

FIG. 1 illustrates a perspective view of an exemplary orthopedic surgical instrument or impactor, in accordance with one or more features of the present disclosure;

FIG. 2 illustrates a perspective view of an example of an impact mechanism contained within the impactor of FIG. 1, in accordance with one or more features of the present disclosure;

FIG. 3a illustrates an exemplary frequency control system, in accordance with one or more features of the present disclosure;

FIG. 3b illustrates an exemplary cycle of a motor/gearbox assembly of the frequency control system shown in FIG. 3a, in accordance with one or more features of the present disclosure;

FIG. 4 illustrates an exemplary process for controlling operational frequency of the impact mechanism shown in FIG. 2, in accordance with one or more features of the present disclosure;

FIG. 5a is a block diagram of an exemplary orthopedic surgical instrument and/or impactor in accordance with one or more features of the present disclosure;

FIG. 5b is a flowchart of an exemplary process for operating of the orthopedic surgical instrument and/or impactor shown in FIG. 5a, in accordance with one or more features of the present disclosure;

FIG. 6 illustrates an exemplary computing apparatus, in accordance with one or more features of the present disclosure;

FIG. 7 illustrates an example of a storage medium to store impactor logic, in accordance with one or more features of the present disclosure; and

FIG. 8 illustrates an example computing platform, in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide a powered, light-weight orthopedic surgical instrument or impactor capable of implementing an impact frequency control mechanism.

### I. Orthopedic Impactor

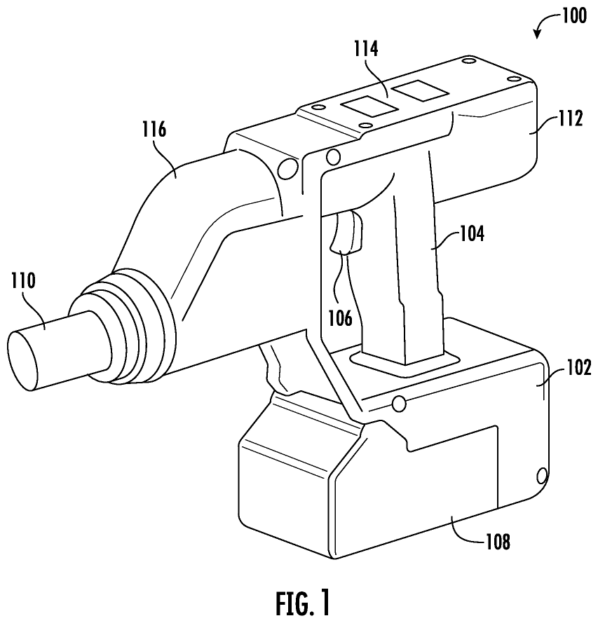

The following description of an orthopedic impactor (e.g., as shown in and discussed in connection with FIGS. 1-2 and 5a-8) is provided here for exemplary, illustrative purposes only and is not intended to limit the current subject matter and/or any of its elements, applications and/or advantages.

Various features or the like of an orthopedic surgical instrument, impactor, or impactor mechanism (terms used interchangeably herein without the intent to limit or distinguish) arranged and configured to transmit a forward energy or motion (e.g., a striking motion, and/or any other motion) to, for example, drive a surgical tool (e.g., a broach) or implant into a patient's bone, and deliver a reverse energy or motion to, for example, remove a struck or lodged surgical tool (e.g., a broach) or implant from a patient's bone will now be described more fully hereinafter with reference to the accompanying drawings, in which one or more features of the orthopedic impactor will be shown and described. It should be appreciated that the various features may be used independently of, or in combination, with each other. It will be appreciated that an orthopedic impactor as disclosed herein may be embodied in many different forms and should not be construed as being limited to the examples set forth herein. Rather, these examples are provided so that this disclosure will convey certain features of the orthopedic impactor to those skilled in the art.

As will be described herein, in accordance with one or more features of the present disclosure, the orthopedic impactor, either alone or in combination with various other features, may be arranged and/or configured to position, insert and/or implant an orthopedic implant such as, for example, but not limited to, an acetabular cup, an intramedullary nail, a femoral hip implant, etc. into a bone matter (e.g., a bone of a patient). Alternatively, or in addition to, the orthopedic impactor may be coupled to a surgical tool such as, for example, but not limited to, a broach, to prepare a bone to receive an orthopedic implant.

In some examples, the orthopedic impactor may be arranged and/or configured to cause application of a forward energy and/or motion to drive an orthopedic implant and/or surgical tool into a patient's bone. In addition, the orthopedic impactor may be arranged and/or configured to cause application of a reverse energy and/or motion to, for example, but not limited to, remove a stuck and/or lodged surgical tool and/or implant from a patient's bone. The orthopedic impactor may be configured to allow selection between the forward and/or reverse application of energy and/or motion by pushing forward and/or pulling back on the orthopedic impactor. During use, a user (e.g., a medical professional, a doctor, a surgeon, etc.) may push forward on an orthopedic impactor that may incorporate the current subject matter's impact mechanism thereby causing a hammer of the impactor to strike a first and/or a forward impaction surface causing the orthopedic impactor to drive an orthopedic implant and/or surgical tool. Alternatively, or in addition, the user may pull back on the orthopedic impactor that may incorporate the current subject matter's impact mechanism thereby causing the impactor's hammer to strike a second and/or a reverse impaction surface causing the orthopedic impactor to produce a reverse impaction to, for example, remove an orthopedic implant or surgical tool.

It should be appreciated that while, for example, the orthopedic impactor may be described herein in connection with driving a broach into a patient's bone to, for example, prepare an intramedullary canal of the patient's bone, the current subject matter is not so limited, and the orthopedic impactor may be used in connection with any surgical tool and/or implant now known and/or hereafter developed. As such, the current subject matter should not be limited to any particular surgical tool, implant, and/or procedure unless explicitly claimed.

In some examples, the orthopedic impactor may be arranged and/or configured to accurately and safely cause application of a force to an orthopedic implant and/or a surgical tool such as, for example, a broach to prepare an intramedullary canal of a patient's bone and/or to assist with removal of the broach from the intramedullary canal of the patient's bone. The orthopedic impactor may be arranged and/or configured to apply a force, while minimizing the risk of injury to the patient and/or to the user's hands during use. In some examples, the orthopedic impactor may be configured to assist its user to deliver a force towards and/or away from a surgical area in, for example, but not limited to, a joint replacement procedure.

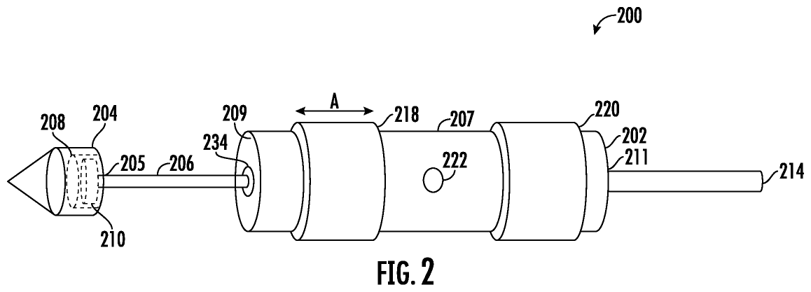

FIG. 1 illustrates an exemplary impactor 100 that may be configured to incorporate and/or house an impact mechanism (as shown and described below with regard to FIG. 2), according to some implementations of the current subject matter. The impactor 100 may be configured to include a housing 102, a handle 104, a trigger assembly 106, a battery 108, a distal connector assembly 110, a motor housing 112 (that may enclose or house a direct current (DC) motor), an electronic display assembly 114, and a distal connector housing 116. The housing 102 may also support any other appropriate components, tools, instruments, etc., such as, for example, but not limited to, sensors, gauges, bone density instruments, feedback components, or any combination thereof. The housing 102 may also feature one or more connection features that can enable the housing to cooperate with and be used in connection with a robotic surgery system. The connection features may be mounting features, input ports for receiving directional instructions, or any combination thereof.

The housing 102 may be configured to house and/or enclose one or more components of the impactor 100. The housing 102 may be manufactured from any suitable material now known or hereafter developed such as, for example, but not limited to, plastic, metal, composite material, fiberglass, and/or any combination thereof. The housing may be designed to be autoclavable and/or, otherwise, sterilizable using any appropriate method.

The housing 102 may include the handle portion 104 with an optional handgrip for comfortable and secure holding of the impactor 100 for use during a procedure (e.g., positioning of an implant into a bone). Alternatively, or in addition to, the housing 102 may incorporate a suitable mounting interface for integrating the impactor 100 into a robotic assembly during use. In some example implementations, the housing 102 may be a unitary structure and/or may include multiple components that may be assembled together.

The housing 102 may also include a reception port for receiving the battery 108. The battery 108 may be a rechargeable battery and may be removed from the housing 102 after use, such as, for example, for recharging. As can be understood, the battery 108 may recharged while coupled to the housing 102. Alternatively, or in addition, the battery may be integrally formed in the housing and rechargeable through the housing. Use of the battery 108 may provide for portability and versatility of the impactor 100 during use, i.e., the user of the impactor 100 does not have to be concerned with power wires (and/or pneumatic tubes) extending from the impactor 100. Alternatively, or in addition, the housing 102 may include one or more power ports (not shown in FIG. 1) that may be used to couple one or more power wires (e.g., to provide power and/or power in addition to the battery power 108) and/or one or more pneumatic tubes (e.g., to provide additional air pressure to the impactor 100 during use). As can be understood, more than one battery 108 may be included in the housing 102 and/or used during procedures. Any type of battery may be used, such as, for example, but not limited to, alkaline, nickel metal hydride (NiMH), lithium ion, and/or any combination thereof. The battery may also be replaceable and/or rechargeable.

The housing 102 may further be configured to include the motor housing 112 that may enclose a DC motor and/or any other type of motor (not shown in FIG. 1) for operation of the impactor 100. The motor may be configured to cause application of a forward movement (e.g., during an implantation procedure) and/or a reverse movement (e.g., during an implant, tool, etc. removal procedure). Alternatively, or in addition to, more than one motor may be included in the housing 102 for performing different operations. For example, one motor may be used for forward operation of the impactor, while another motor may be used for reverse operation of the impactor.

The motor may be actuated using the trigger assembly 106. As can be understood, actuation of the trigger assembly 106 (e.g., depressing the trigger) may actuate the motor (e.g., motor performing rotary movement). Release of the trigger assembly may stop operation of the motor. During use of the impactor 100, a user (e.g., a medical profession, a doctor, a surgeon, etc.) may hold the impactor 100 with their hand using the handle 104 and position one or more of their fingers on the trigger assembly 106, and when they are ready, depress the trigger assembly 106 to begin performing one or more stages of the surgical procedure.

The housing 102 may include the electronic display assembly 114 that may be used to display one or more operating parameters of the impactor 100. These may include, for example, operating power level, power output level, battery level, direction of operation, etc. The assembly 114 may also display one or more alerts to the user, e.g., a fault in the operation, a low power level, etc. Alternatively, or in addition, the electronic display assembly 114 may be optional.

The housing 102 may further include a distal connector housing 116 that may be configured to house the distal connector 110. The distal connector 110 may be configured to have any desired shape (for illustrative, non-limiting purposes only, a cylindrical shape distal connector 110 is shown in FIG. 1). The distal connector 110 may be used for coupling of various tools, instruments, devices, etc. (e.g., drills, cutting tools, effectors, broaches, implants, etc.) that may be used during a procedure. In some example, non-limiting implementations, the distal connector may include a coupling mechanism for coupling such tools, instruments, devices, etc. For example, the coupling mechanism may include a quick-connect mechanism to facilitate exchanges of tools, instruments, devices, etc. Alternatively, or in addition to, the coupling mechanism may selectively couple to an adapter, which may, in turn, couple to one or more tools, instruments, devices, etc.

In some examples, the housing 102 of the impactor 100 may be configured to further house an impact mechanism (as for example, shown in FIG. 2) that may be used to generate forward and/or reverse forces for moving of any tools, instruments, devices, etc. that may be coupled to the impactor 100 for the purposes of performing a procedure. The impact mechanism may be configured as an electro-pneumatic hammer mechanism. The electro-pneumatic hammer mechanism may be configured to power a lightweight compression cylinder in order to compress a column of air (created within the cylinder) and accelerate a weighted striker component of the impact mechanism at a higher speed than the compression cylinder itself. The electro-pneumatic hammer mechanism may be further configured to allow for both forward and reverse impaction based on the direction of hand pressure that is applied to the impactor 100.

FIG. 2 illustrates an impact mechanism 200, according to some implementations of the current subject matter. The impact mechanism 200 may be supported by the housing 102 and, in some exemplary instances, the distal connector housing 116. The impact mechanism 200 may include a housing 202 having a hollow interior for housing a striker mechanism with a striker rod 206 extending outside of the housing 202 and being connected to a distal connector 204. The housing 202 may be configured as, for example, a container (e.g., a cylinder) having a hollow interior. The striker mechanism may be configured to slide within the hollow interior of the housing 202 during operation, e.g., a surgical procedure. The hollow interior of the housing 202 may be enclosed by a wall 207. The wall 207 may be manufactured from any desired material (e.g., metal, plastic, fiberglass, composite, etc.) and may have any desired thickness that may be designed to contain air pressure generated inside the hollow interior of the housing 202 during movement of the striker mechanism.

The housing 202 may include a proximate end 209 and a distal end 211. The proximate end 209 may include an opening 234 in the wall 207. The opening 234 may allow protrusion of as well as forward and reverse translation movement of at least a portion of the striker mechanism's rod during a surgical procedure, which may be caused by the movement of the housing 202 that may be driven by a motor (not shown in FIG. 2). The proximate end 209 may be disposed proximate to the distal connector 204. The distal end 211 may be coupled to a driving rod connector 214 that may, in turn, be coupled to one or more components of the motor assembly (not show in FIG. 2). The opening 234 may be a sealed opening (e.g., using one or more O-ring(s) and/or any other sealing device(s)). The sealing device(s) may be coupled to the wall 207 and may be configured to hermetically seal the opening 234 while the striker mechanism's rod protrudes (and/or translates) through the opening 234. The sealing provided by the sealing device(s) prevents escape of any air from the interior of the housing 202 during operation of the mechanism 200, thereby retaining requisite air pressure for driving the striker mechanism.

The housing 202 may further include one or more compression/decompression chambers 218 and 220. The chambers 218, 220 may be positioned proximate to the ends of the of housing 202. For example, the chamber 218 may be positioned proximate to the proximate end 209 and the chamber 220 may be positioned proximate to the distal end 211. The chambers 218, 220 may be configured as protrusion, extensions, etc. from the surface housing 202, thereby creating an additional interior space to provide for accumulation (and/or compression/decompression) of additional air volume during operation of the mechanism 200, which may be used for movement of the striker mechanism 206.

As shown in FIG. 2, the chambers 218, 220 may have a cylindrical profile; however, as can be understood, the chambers 218, 220 may have any shape and/or form. Moreover, each chamber 218, 220 may have a different shape and/or may be designed to accommodate different volume of air during operation. In some example implementations, one or more chambers 218, 220 may also be configured to be extendible, whereby one or more electro-mechanical mechanisms in the housing 202 may be used to adjust sizes of one or more chambers 218, 220 (by extending away from/contracting toward the surface of the housing 202) so that a different volume of air may be accommodated by each chamber. This may be helpful during operation of the mechanism 200 when greater and/or smaller force may be needed to drive the striker mechanism in one or more directions.

The wall 207 may include one or more openings 222 that may be used as vent opening to allow air to escape during operation of the mechanism 200. As can be understood, one or more openings 222 may be used for venting out air from the interior space of the housing 202. The openings 222 may be temporarily sealable (e.g., using one or more O-rings or other sealing devices). Such temporary sealing may allow maintenance of air volume and pressure in the interior space of the housing 202.

The distal connector 204 may be similar to the distal connector 110 shown in FIG. 1. One or more tools, instruments, devices, etc. may be coupled to the distal connector 204. While the distal connector 204, as illustrated in FIG. 2, may have a conical shape, as can be understood, the distal connector 204 may have any desired shape, size, and/or configuration. It may also be removable from the impact mechanism 200, such as, for the purposes of replacement after being worn out and/or for use during different procedures. The distal connector 204 may also include various coupling mechanisms (e.g., quick-release, friction fit, etc.) for securing tools, instruments, devices, etc. Alternatively, or in addition, the distal connector 204 may serve as a tool, instrument, and/or device.

The distal connector 204 may include a hollow interior connector chamber 208. The chamber may be accessible using an opening 205 positioned at a distal end of the distal connector (the proximal end of the connector 204 may be opposite to its distal end and may serve as an impactor surface, and/or surface for coupling various tools, instruments, devices, etc.). The opening 205 may be configured to accommodate insertion and translation (e.g., unconstrained) movement of the striker mechanism's rod. The chamber 208 may be configured to accommodate positioning and translation (e.g., unconstrained) of a striker hammer 210 that may be coupled to the striker mechanism's rod. During operation of the impact mechanism 200, the striker hammer 210, while being coupled to the striker mechanism's rod, may be configured to translate forward and backward/in-reverse in the interior space of the chamber 208, thereby impacting interior walls of the chamber 208, thereby causing a hammering action of the distal connector 204.

Movement of the housing 202 may be effectuated using a motor of the impactor. This movement may also be performed in a pulsating (or back-and-forth) manner (e.g., similar to a jackhammer) using a predetermined frequency, which may be adjustable, as desired. Further, movement of the housing 202 may be configured to cause movement of the striker mechanism 206 within the interior space of the housing 202. As the housing 202 is moved forward, the striker mechanism 206 may be configured to translate backward toward the distal end 211 of the housing 202. This causes the striker mechanism's rod to further extend into the interior space of the housing 202 (while the opening 234 is sealed). The movement of the housing 202 changes air pressure within the interior space of the housing 202 and in particular, interior spaces of the chambers 218, 220, which creates additional air pressure for driving the striking mechanism 206.

During the reverse motion, the housing 202 may be configured to translate forward toward the proximate end 209. Again, movement of the housing 202 may be effectuated using a motor of the impactor. This movement may also be performed in a pulsating (or back-and-forth) manner (e.g., similar to a jackhammer) using a predetermined frequency, which may be adjustable, as desired. Reverse movement of the housing 202 may likewise be configured to cause movement of the striker mechanism 206 within the interior space of the housing 202. As the housing 202 is moved in reverse, the striker mechanism's rod may further extend from the interior space of the housing 202 (while the opening 234 is sealed). The reverse movement of the housing 202 also changes air pressure within the interior spaces of the chambers 218, 220, which, likewise creates additional air pressure for driving the striking mechanism 206.

In use, the impactor 100 may include any suitable driving mechanism now known or hereafter developed suitable for converting the rotational output from the motor to axial motion. For example, such mechanisms may include a wobble bearing mechanism for driving the impact mechanism 200 in a reciprocating motion, crank-slider mechanism for driving the impact mechanism 200, and/or any other types of mechanisms and/or combination of mechanisms.

### II. Frequency Control of Impact Mechanism

In the existing impactor systems, force and frequency variables associated with an impact are typically dependent on one another. In particular, the force and frequency of impact are dependent on the frequency/rotational speed of the motor of the impactor (which may also include additional variables of potential speed reductions due to gearbox, rotational-to-linear motion conversion mechanism, etc.). This issue is present in impact mechanisms that rely on continuous medium/high operational speed of its motor (e.g., DC, brushless, brushed). Mechanisms that do not rely on medium/high operational speeds typically rely on intermittent (e.g., start/stop, or on/off) operation of the motor, which may put additional load and/or wear-and-tear on motor components and/or require additional power to overcome friction and/or inertia of the mechanism.

In some examples, the current subject matter may be configured to control an impact force of an electro-pneumatic impact mechanism independent of the impact frequency. In particular, the electro-pneumatic impact mechanism may be connected to a drive motor/gearbox via a flywheel and an electromagnetic clutch. This may allow the impact force to be determined by a motor's rotational speed (e.g., consistent with the intended function of the electro-pneumatic impact mechanism), and the impact frequency to be controlled by the energization of the electromagnetic clutch, coupling the impact mechanism to the motor and gearbox.

In some examples, the current subject matter's frequency control mechanism may include a flywheel and electromagnetic clutch, which may be positioned between the output shaft of the motor and the gearbox assembly, and the input shaft of the rotational-to-linear motion conversion mechanism (e.g., a wobble bearing, crank-slider mechanism, as discussed above). The flywheel may be rigidly coupled to the output shaft of the motor and the gearbox, and may have a relatively high moment of inertia. This means that the flywheel may require time and/or torque to spin up to nominal speed but may maintain its angular momentum during operation.

If the flywheel does not have sufficient angular momentum, the friction and inertia of the impact mechanism may greatly slow the motor speed when the electromagnetic clutch is energized and place undesired load on the motor (and, potentially, causing additional wear). The electromagnetic clutch may include an input and an output side that may be magnetically forced together when the clutch is energized and forced apart when it is de-energized (e.g., via a return spring). Torque may be transmitted across the two sides via friction plates and/or small teeth. The flywheel may be rigidly coupled to the input side of the electromagnetic clutch, and the output side of the clutch may be rigidly coupled to the rotational portion of the rotational-to-linear motion conversion mechanism.

The motor may start spinning when a sensor detects a user places their finger over the trigger and pushes/pulls the impact mechanism into the forward and/or reverse position. The motor may stop spinning if the user's finger is removed from the trigger. In some examples, the current subject matter may be pre-programmed with discrete force and/or impact frequency settings. The user may toggle between force and frequency settings independently using buttons and/or other input mechanisms (e.g., sliders, touch screen, switches).

In some examples, the force of impact may be determined by the motor and gearbox speed. The motor and gearbox may also determine the maximum input speed of the rotational-to-linear motion conversion mechanism, and thus, the maximum speed of the compression cylinder and striker. The discrete user input force settings may determine the speed at which the motor (and thus, the gearbox) rotates (e.g., high, medium, low speed settings).

In some examples, impact frequency may be determined by the frequency of energization of the electromagnetic clutch, as set by user input. By way of a non-limiting example, one or more pre-programmed frequencies may be high=15 Hz, medium=6 Hz, low=1 Hz. The user may select one of the pre-programmed frequencies (and/or a frequency may be automatically selected, e.g., based on a specific surgical procedure/application/etc., and/or other settings). When the trigger is depressed, the electromagnetic clutch may energize at the selected frequency (the motor, gearbox, and flywheel, however, may still be spinning at the maximum speed required by the force setting). The duration of energization may be determined by the time taken by the electro-pneumatic impact mechanism to complete one full impact stroke.

In some examples, the energization duration of the clutch may be controlled electronically by sensing a linear position of a compression chamber of the impact mechanism. The clutch may be de-energized when the compression cylinder reaches an initial position.

In some examples, the current subject matter may be advantageous in that the electro-pneumatic impact mechanism (as shown and discussed above with reference to FIG. 2), may be fully utilized with independent force and frequency control while minimizing striker mass and overall weight without compromising impact force.

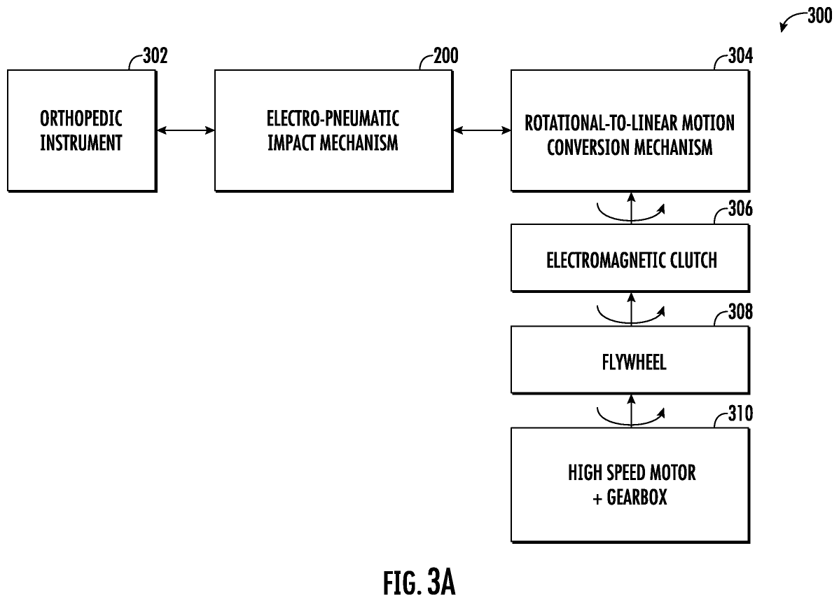

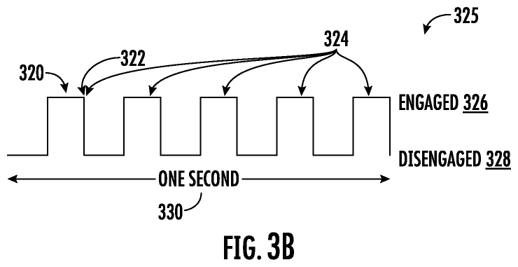

FIG. 3a illustrates an exemplary frequency control system 300, according to some implementations of the current subject matter. The system 300 may include an orthopedic instrument 302, the electro-pneumatic impact mechanism (and/or impact mechanism) 200, a rotational-to-linear motion conversion mechanism (and/or driving mechanism) 304, an electromagnetic clutch 306, a flywheel 308, and a motor and gearbox assembly 310.

As discussed above in connection with FIG. 2, the orthopedic instrument 302 may be coupled to the mechanism 200 using any desired means. In some example implementations, the orthopedic instrument 302 may be coupled to the mechanism 200 using the distal connector 204 (as shown in FIG. 2). The rod connector 214 of the mechanism 200 may, in turn, be used for coupling mechanism 200 to any suitable driving mechanism (e.g., rotational-to-linear motion conversion mechanism) 304 that may be used converting rotational output from the motor of the impactor to an axial and/or translational motion. As stated above, examples of mechanisms 304 may include a wobble bearing mechanism (e.g., driving the mechanism in a reciprocating motion), a crank-slider mechanism, and/or any other types of mechanisms and/or combination of mechanisms.

The electromagnetic clutch 306 may be any existing electromagnetic (EM) clutch device that may be capable of being energized. An exemplary electromagnetic clutch may include a coil, a rotor, an armature plate, and an output side or hub. The output side of the electromagnetic clutch can be coupled to the rotational-to-linear motion conversion mechanism 304 and the rotor can be coupled to the flywheel 308 of the system 300.

Upon actuation and/or energizing (e.g., using a voltage of 24V DC and/or any other voltage) of the electromagnetic clutch 306, the current flows through the coil to generate a strong magnetic field. This causes the rotor to become magnetized and create a magnetic loop to attract the armature plate. The armature plate is then pulled against the rotor, which causes generation of a frictional force upon contact. The armature plate and the output hub are engaged upon acceleration of the load to match the speed of the rotor. The rotor can continue to rotate with the input all the time. When current is removed, the armature plate, under the force of a spring, can be removed from a surface of the rotor. Electromagnetic clutch transmission can occur horizontally and/or vertically to achieve torque transmission, irrespective of high speed and/or no load.

The flywheel 308 may be configured to be rigidly affixed to an output shaft of the motor and gearbox assembly 310. Moreover, the flywheel 308 may be directly mounted to the motor/gearbox side of the electromagnetic clutch 306. The flywheel 308 may be of any known type, and may have a large rotational moment of inertia in order to minimize shock loading of the motor 310.

The motor/gearbox assembly 310 may include a high-speed rotational motor that may be equipped with an encoder (e.g., a rotational position sensor). The encoder of the motor/gearbox assembly 310 may be configured to determine a single operational cycle of the motor/gearbox assembly. A single cycle may correspond to a number of revolutions of the output shaft of the motor/gearbox assembly 310 that it takes to fully translate the mechanism 200 forward then return to its starting position (and/or translate the mechanism 200 backward in reverse mode).

In some examples, the single cycle of the motor/gearbox assembly 310 may be variable. In some example implementations, the single cycle if the assembly 310 may be based on the length of the mechanism 200, a reduction ratio of the rotational/linear motion conversion mechanism 304, and/or any other factors.

Once the single cycle is determined, the duration of the engagement of the electromagnetic clutch 306 may be configured to match the duration of the cycle. A desired frequency (e.g., 5 Hz, 10 Hz, 20 Hz, etc.) may be selected and may be used to determine how often the electromagnetic clutch 306 may engage (without changing the duration of engagement). When the electromagnetic clutch 306 is engaged, the mechanism 200 may be configured to fully cycle and return to the starting position and is then retained in a predetermined position using a retention mechanism (e.g., a ball plunger, a magnet, etc.).

FIG. 3b illustrates an exemplary cycle 325 of the motor/gearbox assembly 310, according to some implementations of the current subject matter. The cycle 325 may be configured to have a length 330 of one (1) second. During the length 300, the motor/gearbox assembly 310 may be configured to alternate between two states: engaged 326 and disengaged 328.

The engaged state 326 may correspond to a state (and/or first and/or engaged state) when the electromagnetic clutch 306 may be configured to provide engagement between the motor/gearbox assembly 310, the flywheel 308, and the rotational-to-linear motion conversion mechanism 304. The disengaged state 328 may correspond to a state (and/or second state and/or disengaged state) when the electromagnetic clutch 306 may be configured to disengage the motor/gearbox assembly 310, the flywheel 308, and the rotational-to-linear motion conversion mechanism 304. The states 326, 328 may be configured to enable movement and/or cycling of the mechanism 200, thereby providing impact.

The cycle 325 may be configured to include one or more engaged/disengaged states 326, 328. As shown in FIG. 3b, there may be five (5) engaged states 326, each engaged state may last during a travel/cycle duration 320 of the mechanism 200, which may last a predetermined period of time. Upon detecting, at 322, that the mechanism 200 has returned to its starting position, the disengaged state 328 may be initiated. Each disengaged state 328 may be configured to occur during another predetermined period of time. In some example implementations, durations of engaged and disengaged states 326, 328 may be equal. Alternatively, or in addition, the durations may be different.

In some examples, the detection, at 322, of a return of the mechanism 200 to its starting position after a cycle may be accomplished using one or more sensors that may be positioned on the housing 202 of the mechanism 200. For example, the sensors may be positioned at or proximate to the proximate end 209 of the mechanism 200 and/or at or proximate to the distal end 211 of the mechanism 200, as shown in FIG. 2. The positioning of the sensors may allow for detection of a specific position of the housing 202 during cycling. Upon detecting of a certain position of the housing 202, the sensors may be configured to report the position to a processor (not shown in FIGS. 2-3b), which may determine whether or not to apply current to the assembly 300 for engagement and/or disengagement of the clutch 306. In some examples, the sensors that may be positioned on the housing 202 may include one or more hall sensors, linear encoder sensors, end stop switches, and/or any other type of translational position sensors.

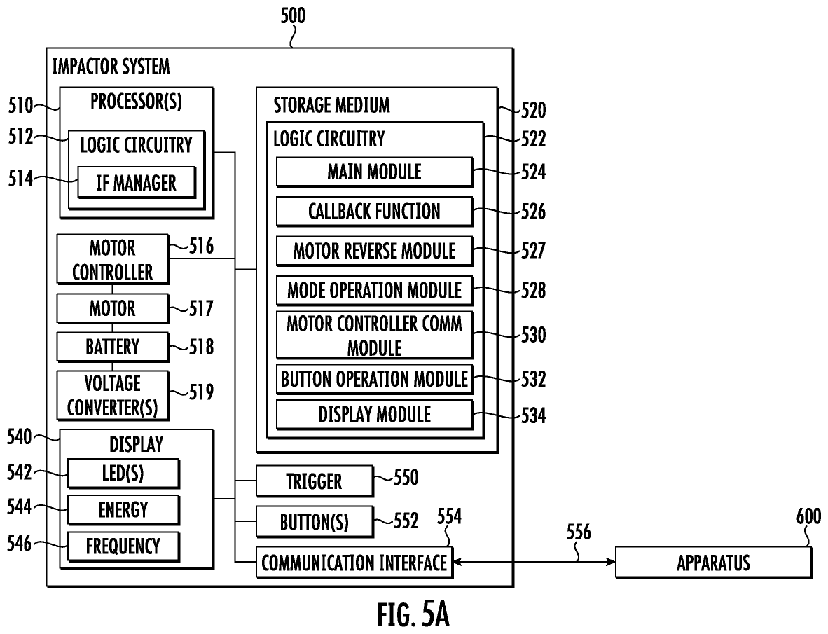

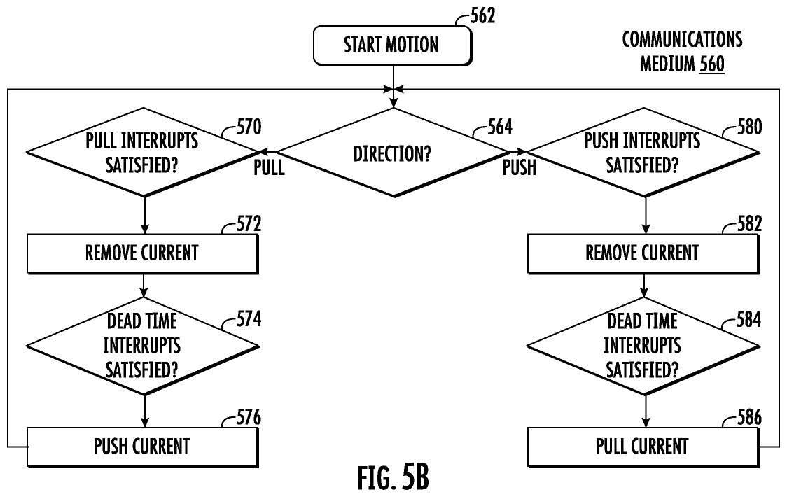

Referring back to FIG. 3b, the number of times engagement/disengagement states 326, 328 occur during a particular cycle 325 may be determined based on a cycling frequency that may be selected (e.g., 5 Hz, 10 Hz, etc.). For example, as illustrated in FIGS. 3b, 5 Hz frequency was selected. Thus, during 1 second cycle period 330, each of the engagement and disengagement states 326, 328 may occur five (5) times.

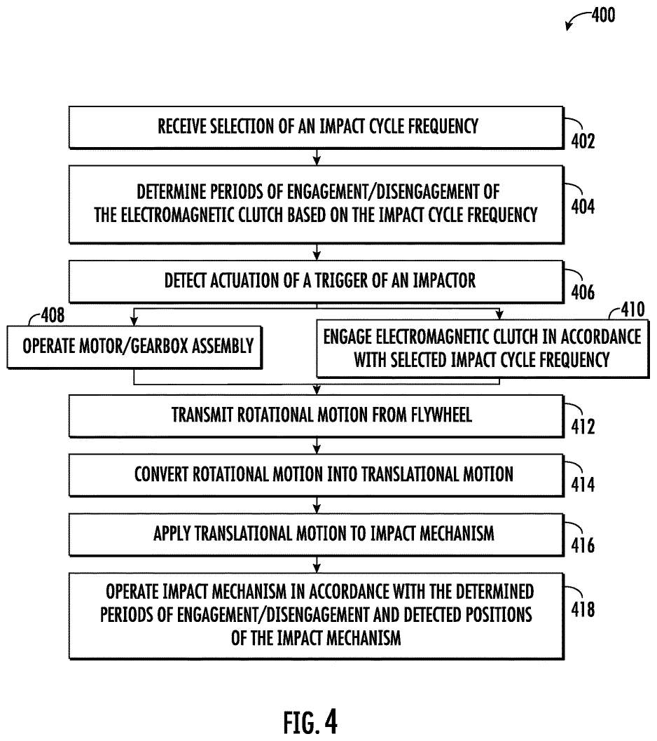

FIG. 4 illustrates an exemplary process 400 for controlling operational frequency of the impact mechanism 200, according to some implementations of the current subject matter. The process 400 may be configured to executed using system 300, as shown in FIGS. 3a-b, that may be implemented in connection with the impactor device 100, as shown in FIG. 1. In particular, one or more processors (e.g., as shown in FIGS. 5a-8) incorporated into the impactor device 100 may be used to control application of current to one or more components of the system 300.

At 402, a selection of a frequency of impact may be received. By way of a non-limiting example, the impact frequency may be 5 Hz, 10 Hz, 15 Hz, and/or any other desired frequency. The selected frequency may be used to determine the length of and a number of single impacts (e.g., forward and/or reverse motions) by the mechanism 200. The selection of such frequency may be performed automatically by the impactor 100 and/or manually by the user of the impactor 100. In some examples, a length of a single cycle may also be selected and/or predetermined. For example, a length of a single cycle may be one (1) second, as shown in FIG. 3b. As can be understood, any length of a single cycle may be selected/predetermined.

Using the information related to the selected frequency of impact as well as the cycle length, the impactor's processor may be configured to determine one or more periods of engagement 326 and/or disengagement 328 of the electromagnetic clutch 306, at 404. As shown in FIG. 3b, each period of engagement/disengagement 326, 328 may have a predetermined duration. Duration of such periods may be selectable and/or predetermined in accordance with the length of a cycle and impact frequency. Further, the impact frequency and cycle length may be used to determine a number of such periods of engagement/disengagement 326, 328. Alternatively, or in addition, a position of the impact mechanism 200 (as, for example, determined by the sensors positioned on its housing 202) may be used to determine when engagement and/or disengagement of the electromagnetic clutch 306 may need to occur. For example, a complete back-and-forth translational motion of the impact mechanism 200 (during the time when the electromagnetic clutch 306 is engaged) may be used to signal disengagement of the electromagnetic clutch 306.

At 406, the system 300 may be configured to detect actuation of the trigger 106 of the impactor 100. This may be accomplished by the user of the impactor 100 placing user's finger on the trigger 106 and squeezing it. One or more sensors in the impactor 100 may be used to detect such squeezing and transmit one or more corresponding signals to the impactor's processor.

Upon actuation of the trigger 106, the motor/gearbox assembly 310 may be configured to begin operating (e.g., spinning), at 408, and/or actuation of the electromagnetic clutch 306 may be configured to occur. The actuation of the electromagnetic clutch 306 may be in accordance with the selected impact cycle frequency (as received at 402) and/or determined periods of engagement/disengagement (as determined at 404).

Once the electromagnetic clutch 306 is engaged, it may provide a clutching connection between the flywheel 308, the motor/gearbox assembly 310, and the rotational-to-linear motion conversion mechanism 304. In particular, the electromagnetic clutch may be configured to provide for transmission of rotation motion from the flywheel 308 to the conversion mechanism 304, at 412.

The conversion mechanism 304 may then be configured to convert transmitted rotational motion into translational motion, at 414. As discussed above, the conversion mechanism 304 may be any known mechanism, such as, for example, a wobble bearing mechanism, a crank-slider mechanism, and/or any other types of mechanisms and/or combination of mechanisms. The generated translational motion may be applied to the impact mechanism 200 for generation of an impact, at 416. The impact mechanism 200 may then be operated in a cyclic forward-and-back motion (and/or cyclic back-and-forward motion (corresponding to a reverse impact)), at 418.

Operation of the impact mechanism 200 and, in particular, its positional movements may be monitored by one or more sensors disposed on the housing 202 of the impact mechanism 200. As discussed above, one of the sensors may be positioned at or proximate to the proximate end 209 of the housing 202 and the other sensor may be positioned at or proximate to the distal end 211 of the housing 202. In a forward impact motion, the impact mechanism 200 may be configured to translate forward and, upon completing of an impact, return to its original position. Once the impact mechanism 200 has returned to its original position, the sensors may be configured to transmit one or more signals to the impactor's processor to indicate that the impact cycle has been completed and thus, the electromagnetic clutch 306 may be temporarily disengaged, e.g., enter into the disengagement state 328.

In accordance with the selected frequency (e.g., 5 Hz, 10 Hz, etc.), the cycle length (e.g., 1 second), and the number of states 326, 328, the electromagnetic clutch 306 may, again, be engaged causing movement of the impact mechanism 200 during the engagement state 326. As discussed above, alternation between as well as length of each engagement and disengagement states 326, 328 may be governed by the frequency, cycle length and/or the number of states 326, 328 As can be understood, both engagement/disengagement of the components of the system 300 may be similar during forward and reverse impact operation of the impactor 100 and the impact mechanism 200. The cycle length as well as frequency may also be selectable in accordance with a specific application and/or use of the impactor 100.

### III. Impactor System

FIG. 5a is a block diagram of an exemplary orthopedic surgical instrument and/or impactor 500, according to some implementations of the current subject matter. FIG. 5b is a flowchart of an exemplary process 560 for operating of the orthopedic surgical instrument and/or impactor 500 shown in FIG. 5a, according to some implementations of the current subject matter. The impactor 500 may be similar to the impactor shown and described above in connection with FIGS. 1-4.

The impactor 500 may combine with any suitable example of the systems, devices, and methods disclosed herein. The impactor 500 may include processor(s) 510, a non-transitory storage medium 520, a motor controller 516, a motor 517, a battery 518, a voltage converter 519, a display 540, a trigger 550, button(s) 552, and a communication interface 554. The processor(s) 510 may include one or more processors, such as a programmable processor, a micro-controller unit (MCU), and/or the like. The processor(s) 510 may include processing circuitry to implement impactor logic circuitry 512 and 522.

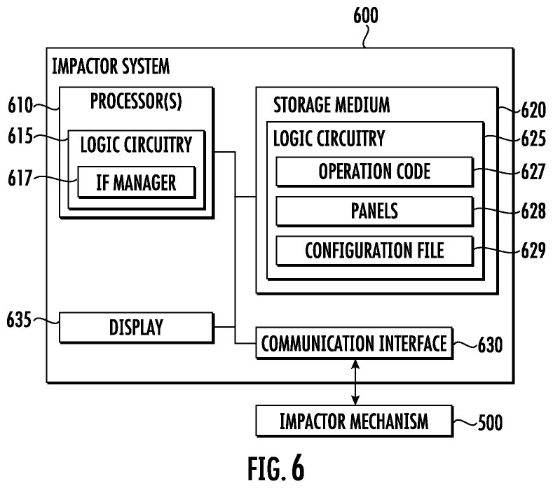

The processor(s) 510 may operatively couple with a non-transitory storage medium 520. The non-transitory storage medium 520 may store logic, code, and/or program instructions executable by the processor(s) 510 for performing one or more operations including the operations of the impactor logic circuitry 522. The non-transitory storage medium 520 may include one or more memory units (e.g., fixed or removable media or external storage such as a flash memory, secure digital (SD) card, random-access memory (RAM), read only memory (ROM), a flash drive, a hard drive, a solid-state drive (SSD) and/or the like). The memory units of the non-transitory storage medium 520 can store logic, code and/or program instructions executable by the processor(s) 510 to perform any suitable implementations of the current subject matter, as described herein. For example, the processor(s) 510 may execute instructions such as instructions of impactor logic circuitry 525 causing the motor 517 to operate the impact mechanism 200 shown and described in connection with FIGS. 2-4 at an impact energy and/or frequency selected by a user via button(s) 552 and/or via apparatus 600 (as shown in FIG. 6).

The processor(s) 510 may include code for the impactor 500 in memory within the processor(s) 510 and/or closely connected such as flash memory. The impactor logic circuitry 512 may represent code in or near the processor(s) 512 for execution by the processor(s) 510 and may include a user interface manager 514. The user interface manager 514 may include code executing on the processor(s) 510 to detect and respond to user input as well as to detect the motor controller 516 (such as, for example, a Maxon EPOS4 Controller) and establish communication with the motor controller 516.

The user interface manager 514 may communicate with the motor controller 516 to receive status information about the motor 517 and to control operation of the motor 517. For instance, all button presses of button(s) 552 and edit events may be posted to the user interface manager 514 and processed in real-time. The user interface manager 514 may communicate commands with the motor controller 516 to execute in response to the user's actions via button presses, system states, and error conditions. The user interface manager 514 may communicate alerts, warnings, and notifications to a user via the display 540 and or the apparatus 600 (as shown in FIG. 6) via the communications interface 554. Further, the user interface manager 514 may also handle user's response to alerts.

The motor 517 may include a DC motor, and/or any other motor. The battery 518 may include any desired power source.

The voltage converter(s) 519 may include a DC-DC voltage converters to adjust the voltage of signals to various voltages required to operate the components of the impactor 500 such as the processor(s) 510, the storage medium 520, and motor controller 516, the display 540, the trigger 550, the buttons 552, the communications interface 554, and/or the like.

The storage medium 520 may include a code for execution by the processor(s) 510 to operate the impactor 500. If desired, the processor(s) 510 may copy code from the storage medium 520 to memory closer to the processor(s) 510 to facilitate faster execution of the code. For instance, the user interface manager 514 may include code copied from the impactor logic circuitry 522 to memory closer to the processor(s) 510 for execution.

The impactor logic circuitry 522 may include code for operation of the impactor 500 stored in hardware of the storage medium such as volatile or non-volatile memory in the storage medium 520. The impactor logic circuitry 522 may include a main module 524, a callback module 526, a motor reverse module 527, a mode operation module 528, a motor controller communications module 530, a button operation module 532, and a display module 534.

The main module 524 may include setup and loop functions. The setup function may run once at start-up and the loop function may run continuously afterwards. The setup function may attach interrupts that run when button(s) 552 are pressed on the user interface, initializes Timer1 which runs the trigger interrupt service routine (ISR), and initializes an impact delay for the motor 517. The loop function allows the motor 517 to operate in the user-desired mode when the trigger 550 is enabled and pulled. The loop function also handles showing the user that the trigger state is enabled via LED(s) 542 of the display 540 and/or via the apparatus 600 (shown in FIG. 6).

The callback function 526 may be, e.g., an ISR that runs every millisecond. In some example implementations, the callback function 526 may run periodically with at a time period of more than one millisecond or less than one millisecond.

The motor reverse module 527 may include functions to prepare to reverse the motor 517, motor direction change of the motor 517, calculate impact delay of the mechanism 200, and setup flutter time delays to set the frequency of impact while in flutter mode. These functions may switch the direction of the motor 517, reversing the motor 517 to allow for bi-directional operation of the mechanism 200, and may also determine the delay between reversals for controlling a frequency of impacts of the mechanism 200 in a flutter mode.

The mode operation module 528 may include the functions of position check, flutter check, and oscillation check functions which are called for normal/full-swing mode, high-frequency/flutter mode, and oscillation mode respectively. Normal operation checks the position of the motor 517 then calls the prepare to reverse function. In some examples, the position of the motor 517 may be monitored via an encoder on a shaft of motor 517 that produces a count responsive to increments of rotation of the stator or shaft of the motor 517.

The motor controller communication module 528 may include the functions of enable motor controller 516 functions, set motor amperage (upper bound amperage), zero motor amperage (lower bound amperage), and disable the motor controller 516 functions. These functions communicate to the motor controller 516 whether or not to operate the motor 517 as well as set the operating amperage bounds for the motor 517.

The button operation module 532 may include functions to handle setting user-desired amperage and frequency to operate the motor 517 in addition to setting the operation mode and enabling the trigger 550. The functions may include energy plus to increase the energy of impact by the mechanism 200, energy minus to increase the energy of impact by the mechanism 200, frequency plus to increase the frequency of impacts by the mechanism 200, frequency minus to decrease the frequency of impacts by the mechanism 200, select operating mode to switch between available modes of operation (e.g., full-swing mode, flutter mode, or oscillation mode), and set trigger state to enable or disable the trigger 550. In some examples, these functions may be accessed via the apparatus 600 (shown in FIG. 6) and/or the button(s) 552. In alternate implementations, a touch screen may be included in the display in lieu of or in addition to the button(s) 552.

The display module 534 may include functions handle the logic for displaying the amperage and frequency on the user interface. The functions may include energy display and frequency display.

The display 540 may include LED(s) 540 and numerical, alphanumeric, or graphical displays such as LED displays or liquid crystal displays (LCDs) to present a number representative of the energy 544 and frequency 546 selected for operation of the motor 517. The button(s) 552 may include one or more buttons located in the display 540 and, In some examples, adjacent to the energy 544 and frequency 546 displays to provide a user with an interface to increase and/or decrease the energy and/or frequency of the impact of the mechanism 200 on the forward and/or the reverse motion.

The trigger 550 may include a trigger or other button or switch that, when actuated, can cause the impactor 500 to operate if the trigger 550 is enabled. If the trigger 550 is disabled, depressing the trigger 550 may not cause the impactor 500 to operate. In some examples, the trigger 550 cannot be depressed when the trigger 550 is disabled.

The processor(s) 510 may couple to a communication interface 554 to communicate with an apparatus 600 via a communications medium 556. The communications medium 556 may comprise a wired or wireless interface to communicatively coupled the impactor 500 with the apparatus 600 shown in FIG. 6.

The communication interface 554 may communicate user commands to and/or from the apparatus 600 to the impactor 500 to operate the impactor 500 via the functionality described in conjunction with the impactor 500. In some examples, the apparatus 500 may operate the motor 517 in addition to configuring parameters of operation of the motor 517 such as the upper current bound, the lower current bound, the operating current, the upper frequency bound, the lower frequency bound, the operating frequency, the mode of operation of the motor 517, and/or the like. In some examples, the communication interface 554 may communicate information about the operation of the impactor 500 to the apparatus 600 such as the energy of operation, the frequency of operation, the mode of operation, events or alerts associated with the impactor 500, and log information such as time and date of use, impact detections, encoder counts, and/or the like.

The communication interface 556 (and similarly, communication interface 630 shown in FIG. 6) may include circuitry to transmit and receive communications through a wired and/or wireless media such as an Ethernet interface, a wireless fidelity (Wi-Fi) interface, a cellular data interface, and/or the like. In some examples, the communication interface 556 (and/or interface 630) may implement logic such as code in a baseband processor to interact with a physical layer device to transmit and receive wireless communications from apparatus 600. For example, the communication interface 556 (and/or interface 630) may implement one or more of local area networks (LAN), wide area networks (WAN), infrared, radio, Wi-Fi, point-to-point (P2P) networks, telecommunication networks, cloud communication, and the like.

Referring to FIG. 5b, at 562, the process 560 may be initiated by starting motion of the motor. The motion may be started in any direction, at 564, either pulling the mechanism 200 and/or pushing the mechanism 200.

In some examples, the impactor logic circuitry may monitor for a reduction in the count below a threshold or by a threshold deceleration of the counts. In some examples, the counts may vary based on a gear ration of the gear box coupled with the motor 517. The gear ratio may affect the granularity of the stator movement of the motor 517 per count, reducing the number of counts per stator rotation for gear boxes with low gear ratios such as 4.8:1 as compared with the number of counts per stator rotation for gear boxes with higher gear ratios such as 14:1. In such implementations, a threshold count may be different depending on the gear ratio of the gear box connected to the motor 517.

The impactor logic circuitry may determine if the number of interrupts received during pulling the hammer represent the selected number of interrupts, at 570. In some examples, the movement of the motor may be closely coupled with the movement of the mechanism 200. The number of interrupts may represent the counts from the encoder of the motor or may represent counts of clock cycles so the impactor logic circuitry may determine whether the counts received at impact are within an expected range of counts for impact of the mechanism 200 on the reverse. If the counts differ from the expected range of the counts, an event and/or alert may be generated to indicate a potential problem with the impactor 500.

If the number of interrupts are satisfied, the impactor logic circuitry may remove current from the motor 517, at 572 for a delay time (or dead time), at 574 that adjusts the frequency of impact of the hammer to a user selected frequency. The interrupts may represent clock cycles of a clock or may represent counts of stator movement determined to represent the amount of delay to include at the time of reversal of the motor 517 to set the frequency of impacts at the frequency selected by the user. In some examples, the interrupts may represent the callback function such as the callback function 526 shown in FIG. 5 a and may, e.g., have a period of 1 millisecond. In other implementations, the period may be less than one millisecond or more than one millisecond.

Once the delay time is satisfied, the impactor logic circuitry may apply a push current to the motor 517, at 576, to rotate the stator of the motor and the shaft of the motor 517 in the opposite direction to push the mechanism 200 forward. For example, In some examples, a 9-ampere current may be a low energy setting and a 20-ampere current may be a high energy setting.

After applying the push current, the process 560 may return to 580 via 564. At 580, the impact logic circuitry may determine if the number of interrupts received at impact are within an expected range of counts for impact of the mechanism 200 on the forward impact. If the counts differ from the expected range of the counts, an event and/or alert may be generated to indicate a potential problem with the impactor 500.

If the number of push interrupts are satisfied, the impactor logic circuitry may remove current from the motor 517, at 582, for a delay time (or dead time), at 584 to adjust the frequency of impact of the hammer on the forward impact surface based on a user selected frequency. The interrupts may represent clock cycles of a clock or may represent counts of stator movement determined to represent the amount of delay time required at the time of reversal of the motor 517 to set the frequency of impacts at the frequency selected by the user. In some examples, the interrupts may represent the callback function such as the callback function 526 shown in FIG. 5a and may, e.g., have a period of 1 millisecond. In other implementations, the period may be less than one millisecond or more than one millisecond.

Once the delay time is satisfied, the impactor logic circuitry may apply a pull current to the motor 517, at 586, to rotate the stator of the motor and the shaft of the motor 517 in the opposite direction to pull the mechanism 200 backwards towards the reverse impact.

FIG. 6 illustrates an exemplary computing apparatus 600, according to some implementations of the current subject matter. The apparatus 600 may be a computing device that may be communicatively coupled with an orthopedic surgical instrument or impactor such as, orthopedic impactor 500 (e.g., as shown in FIG. 5a). The apparatus 600 may be a computer in the form of a smart phone, a tablet, a notebook, a desktop computer, a workstation, or a server. The apparatus 600 can combine with any suitable example of the systems, devices, and methods disclosed herein. The apparatus 600 can include processor(s) 610, a non-transitory storage medium 620, communication interface 630, and a display 635. The processor(s) 610 may comprise one or more processors, such as a programmable processor (e.g., a central processing unit (CPU)). The processor(s) 610 may comprise processing circuitry to implement impactor logic circuitry 615 such as the impactor logic circuitry 512 shown in FIG. 5a.

The processor(s) 610 may include memory such as flash memory to contain program code for execution by the processor(s) 610. In some examples, the processor(s) 610 may have random access memory to contain a copy of code from flash memory or read only memory to facilitate faster execution of code. In some examples, the processor(s) 610 may include cache to contain data for faster calculations or execution. In some examples, the processor(s) 610 may include an impactor logic circuitry 615, which may include a user interface manager 617. The user interface manager 617 may function as a state machine controlled by keypad inputs, internal events or alarms, boundary conditions, exceptions and supervisory input to the user interface manager 617. The user interface manager 617 may process button presses and may update a main screen on the display 635 reflecting the state of the application.

Upon startup of the user interface manager 617, a handler may be installed to detect the motor controller 516 of the impactor 500 and to establish communication with the motor controller 516. In some examples, the button presses of button(s) 552 and edit events may be posted to a panel in the display 635 and may be processed in real-time. Motor controller commands may be executed upon the user's actions via button presses, system states, and error conditions. Further, the user interface manager 617 may implement alerts, warnings, and notifications and display the alerts, warnings, and notifications via the display 635. The user interface manager 617 may also include code to handle the user's response to alerts, warnings, and notifications.

The processor(s) 610 may operatively couple with a non-transitory storage medium 620. The non-transitory storage medium 620 may store logic, code, and/or program instructions executable by the processor(s) 610 for performing one or more instructions including the impactor logic circuitry 625. The non-transitory storage medium 620 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 620 may store logic, code and/or program instructions executable by the processor(s) 610 to perform any suitable implementation of the methods described herein. For example, the processor(s) 610 may execute instructions such as instructions of impactor logic circuitry 625 causing one or more processors of the processor(s) 610 to communicate user commands to an impactor 500 (as shown in FIG. 5a) and/or to communicate events, alerts, operation parameters for the impactor 500, and configurations.

The impactor logic circuitry 625 may include operation code 627, panels 628, and a configuration file 629. The operation code 627 may include functionality to set energy boundaries for operation of the impactor 500, set frequency boundaries for operation of the impactor 500, set an operating energy, set an operating frequency, set a mechanism 200 detection profile, set a boundary for a push current interrupt count, set a boundary for a pull current interrupt count, set a delay time or dead time interrupt count to establish a frequency of impact, set an operating mode (full swing, flutter, or oscillation), and/or the like.

The panels 628 may define graphical user interfaces for display of information and for receiving input parameters or configurations from a user. The configuration file 630 may include user selected parameters such as a motor controller with which to communicate, boundaries for energy (current), boundaries for frequency of impact, numbers of interrupts expected for push current and for pull current, and/or number of interrupts to receive to establish a frequency of impact.

The processor(s) 610 may couple to a communication interface 630 to transmit the data, code, or commands to and/or receive data, code, or commands from one or more external devices (e.g., a terminal, display device, a smart phone, a tablet, a server, or other remote device). The communication interface 630 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some examples, the communication interface 630 may implement logic such as code in a baseband processor to interact with a physical layer device to transmit and receive wireless communications from the impactor 500. For example, the communication interface 630 may implement one or more of local area networks (LAN), wide area networks (WAN), infrared, radio, Bluetooth, Wi-Fi, point-to-point (P2P) networks, telecommunication networks, cloud communication, and the like.

The processor(s) 610 may couple to a display 630 to display panels 628 for a user interface and/or other user interface items such as a message or notification via, graphics, video, text, and/or the like. In some examples, the display 630 may include a display on a terminal, a display device, a smart phone, a tablet, a server, or a remote device.

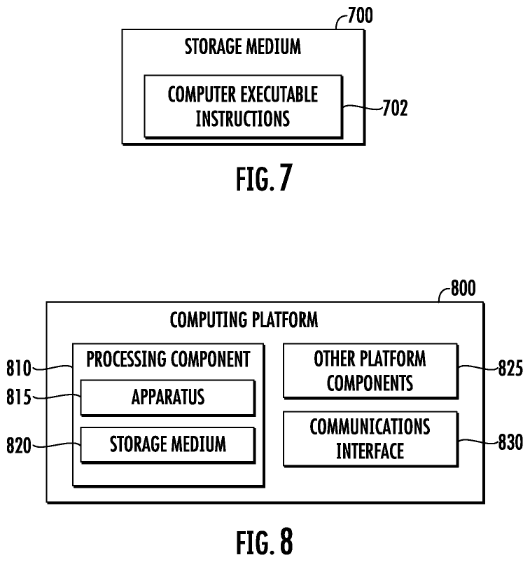

FIGS. 7-8 illustrate example implementations of a storage medium and computing platform for an orthopedic surgical instrument or impactor in accordance with one or more features of the present disclosure. FIG. 7 illustrates an example of a storage medium 700 to store impactor logic. Storage medium 700 may include an article of manufacture. In some examples, storage medium 700 may include any non-transitory computer readable medium or machine readable medium, such as an optical, magnetic or semiconductor storage. Storage medium 700 may store various types of computer executable instructions 702, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 8 illustrates an example computing platform 800. In some examples, as shown in FIG. 8, the computing platform 800 may include a processing component 810, other platform components or a communications interface 830. According to some examples, computing platform 800 may be implemented in a computing device such as a server in a system such as a data center or server farm that supports a manager or controller for managing configurable computing resources as mentioned above. Furthermore, the communications interface 830 may include a wake-up radio (WUR) and may be capable of waking up a main radio of the computing platform 800.

According to some examples, processing component 810 may execute processing operations or logic for apparatus 815 described herein such as the impactor logic circuitry 512, 615, and 625 illustrated in FIGS. 5a-b. Processing component 810 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 820, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 825 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 830 may include logic and/or features to support a communication interface. For these examples, communications interface 830 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 800 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 800 described herein, may be included or omitted in various implementations of computing platform 800, as suitably desired.

The components and features of computing platform 800 may be implemented using any combination of discrete circuitry, ASICs, logic gates and/or single chip architectures. Further, the features of computing platform 800 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 800 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the claimed subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this Detailed Description by this reference, with each claim standing on its own as a separate implementation of the present disclosure.

1. An orthopedic impactor, comprising:
a motor assembly;
an electromagnetic clutch;
a rotational-to-linear conversion mechanism coupled to the motor assembly using the electromagnetic clutch; and
an impact mechanism coupled to the rotational-to-linear conversion mechanism;
wherein the electromagnetic clutch is configured to transmit a rotational motion generated by the motor assembly to the rotational-to-linear conversion mechanism to convert the rotational motion to a translational motion and to cause one or more cyclic translations of the impact mechanism during a predetermined period of time.

2. The orthopedic impactor of claim 1, wherein the cyclic translations of the impact mechanism include at least one of: one or more oscillatory forward movements, one or more reverse movements, and any combinations thereof.

3. The orthopedic impactor of claim 1, wherein the electromagnetic clutch is configured to at least one of: temporarily engage and temporarily disengage the motor assembly and the rotational-to-linear conversion mechanism.

4. The orthopedic impactor of claim 1, further comprising a flywheel coupled to the motor assembly, the flywheel is configured to transmit the rotational motion to the rotational-to-linear conversion mechanism upon engagement of the electromagnetic clutch.

5. The orthopedic impactor of claim 1, wherein the impact mechanism includes one or more sensors configured to detect a position of the impact mechanism.

6. The orthopedic impactor of claim 5, wherein the electromagnetic clutch is configured to be at least one of: engaged and disengaged, based on the position of the impact mechanism.

7. The orthopedic impactor of claim 5, wherein at least one of: an engagement and a disengagement of the electromagnetic clutch is determined based on at least one of the following: a length of a cycle of operation of the impact mechanism, a frequency of operation of the impact mechanism, a number of engagement and/or disengagement states during the cycle of operation of the impact mechanism, and any combination thereof.

8. The orthopedic impactor of claim 1, wherein:
the impact mechanism is configured to operate in at least one of: a forward translational motion, a reverse translational motion, and any combination thereof;
in the forward translational motion, the electromagnetic clutch is configured to:
provide an engagement of the motor assembly during the forward translational motion of the impact mechanism from a forward initial position to a forward impact position, and
disengage the motor assembly upon the impact mechanism returning to the forward initial position; and
in the reverse translational motion, the electromagnetic clutch is configured to:
provide an engagement of the motor assembly during the reverse translational motion of the impact mechanism from a reverse initial position to a reverse impact position, and
disengage the motor assembly upon the impact mechanism returning to the reverse initial position.

9. (canceled)

10. (canceled)

11. A method, comprising:
receiving, using at least one processor, a selection of an impact cycle frequency for operating of an impact mechanism of an impactor;
determining, using the at least one processor, based on the impact cycle frequency, one or more periods of engagement and/or disengagement of an electromagnetic clutch of the impactor;
detecting, using the at least one processor, an actuation of a trigger of the impactor;
triggering, using the at least one processor, at least one of: an operation of a motor of the impactor and an engagement of the electromagnetic clutch using the impact cycle frequency; and
operating, using the at least one processor, the impact mechanism of the impactor using the one or more periods of engagement and/or disengagement and one or more positions of the impact mechanism.

12. The method of claim 11, wherein the electromagnetic clutch is configured to transmit rotational motion resulting from the operation of the motor from a flywheel of the impactor to a conversion mechanism, the conversion mechanism is configured to convert the rotational motion to a translational motion of the impact mechanism causing the impact mechanism to perform one or more cyclic translations of the impact mechanism during a predetermined period of time.

13. The method of claim 12, wherein the cyclic translations of the impact mechanism include at least one of: one or more oscillatory forward movements, one or more reverse movements, and any combinations thereof.

14. The method of claim 11, further comprising:
causing, using the at least one processor, the electromagnetic clutch to at least one of: temporarily engage and temporarily disengage the motor assembly and the rotational-to-linear conversion mechanism.

15. The method of claim 11, wherein the impact mechanism includes one or more sensors configured to detect the one or more positions of the impact mechanism; and
wherein the electromagnetic clutch is configured to be at least one of: engaged and disengaged, based on the position of the impact mechanism.

16. (canceled)

17. The method of claim 15, wherein at least one of: an engagement and a disengagement of the electromagnetic clutch is determined based on at least one of the following: a length of a cycle of operation of the impact mechanism, a frequency of operation of the impact mechanism, a number of engagement and/or disengagement states during the cycle of operation of the impact mechanism, and any combination thereof.

18. The method of claim 11, wherein:
the impact mechanism is configured to operate in at least one of: a forward translational motion, a reverse translational motion, and any combination thereof;
in the forward translational motion, the electromagnetic clutch is configured to:
provide an engagement of the motor assembly during the forward translational motion of the impact mechanism from a forward initial position to a forward impact position, and
disengage the motor assembly upon the impact mechanism returning to the forward initial position; and
in the reverse translational motion, the electromagnetic clutch is configured to:
provide an engagement of the motor assembly during the reverse translational motion of the impact mechanism from a reverse initial position to a reverse impact position, and
disengage the motor assembly upon the impact mechanism returning to the reverse initial position.

19. (canceled)

20. (canceled)

21. A system, comprising:
at least one processor; and
at least one non-transitory storage media storing instructions, that when executed by the at least one processor, cause the at least one processor to:
receive a selection of an impact cycle frequency for operating of an impact mechanism of an impactor;
determine, based on the impact cycle frequency, one or more periods of engagement and/or disengagement of an electromagnetic clutch of the impactor;
detect an actuation of a trigger of the impactor;
trigger at least one of: an operation of a motor of the impactor and an engagement of the electromagnetic clutch using the impact cycle frequency; and
operate the impact mechanism of the impactor using the one or more periods of engagement and/or disengagement and one or more positions of the impact mechanism.

22. The system of claim 21, wherein the electromagnetic clutch is configured to transmit rotational motion resulting from the operation of the motor from a flywheel of the impactor to a conversion mechanism, the conversion mechanism is configured to convert the rotational motion to a translational motion of the impact mechanism causing the impact mechanism to perform one or more cyclic translations of the impact mechanism during a predetermined period of time.

23. The system of claim 22, wherein the cyclic translations of the impact mechanism include at least one of: one or more oscillatory forward movements, one or more reverse movements, and any combinations thereof.

24. The system of claim 21, wherein the at least one processor is configured to:
cause the electromagnetic clutch to at least one of: temporarily engage and temporarily disengage the motor assembly and the rotational-to-linear conversion mechanism.

25. The system of claim 21, wherein the impact mechanism includes one or more sensors configured to detect the one or more positions of the impact mechanism; and
wherein the electromagnetic clutch is configured to be at least one of: engaged and disengaged, based on the position of the impact mechanism.

26. (canceled)

27. The system of claim 25, wherein at least one of: an engagement and a disengagement of the electromagnetic clutch is determined based on at least one of the following: a length of a cycle of operation of the impact mechanism, a frequency of operation of the impact mechanism, a number of engagement and/or disengagement states during the cycle of operation of the impact mechanism, and any combination thereof.

28. The system of claim 21, wherein:
the impact mechanism is configured to operate in at least one of: a forward translational motion, a reverse translational motion, and any combination thereof;
in the forward translational motion, the electromagnetic clutch is configured to:
provide an engagement of the motor assembly during the forward translational motion of the impact mechanism from a forward initial position to a forward impact position, and
disengage the motor assembly upon the impact mechanism returning to the forward initial position; and
in the reverse translational motion, the electromagnetic clutch is configured to:
provide an engagement of the motor assembly during the reverse translational motion of the impact mechanism from a reverse initial position to a reverse impact position, and
disengage the motor assembly upon the impact mechanism returning to the reverse initial position.

29. (canceled)

30. (canceled)

31-40. (canceled)
